(19) United States
(12) Patent Application Publication
(10) Pub. No.: US 2024/0390414 A1
(43) Pub. Date: Nov. 28, 2024

(54) USE OF SODIUM THIOSULFATE (STS) WITH AND WITHOUT COFACTORS FOR THE TREATMENT OF INFLAMMATORY, NEURODEGENERATIVE AND NEUROPSYCHIATRIC CONDITIONS

(71) Applicant: Thiogenics LLC, Troy, MI (US)

(72) Inventors: Christian Bogner, Birmingham, MI (US); Alex Zaharakis, Tucson, AZ (US)

(21) Appl. No.: 18/208,186

(22) Filed: Jun. 9, 2023

Related U.S. Application Data
(60) Provisional application No. 63/468,225, filed on May 22, 2023.

(51) Int. Cl. A61K 33/04; A61K 45/06

(52) U.S. Cl. CPC A61K 33/04; A61K 45/06; A61K 9/0014; A61K 9/0043; A61K 9/0053

(57) ABSTRACT

Disclosed is a method for treating oxidative stress or inflammation which comprises administering to a subject suffering from oxidative stress or inflammation a therapeutically effective amount of sodium thiosulfate.

### CROSS REFERENCE TO RELATED APPLICATION

This application claims benefit of U.S. Provisional Application Ser. No. 63/468,225 filed May 22, 2023, the entire disclosure of which is incorporated herein by reference.

### BACKGROUND AND SUMMARY

Sodium thiosulfate (STS) pentahydrate has numerous industrial applications including such uses as removing chlorine from solutions, bleaching paper pulp, and extracting silver from ores. It is also used as a fixer in photography, a mordant in dyeing and printing textiles, and as a pharmaceutical ingredient. Even though thousands of metric tons of sodium thiosulfate pentahydrate are produced annually, only a few hundred kilograms are utilized pharmaceutically for the production of sodium thiosulfate injection, as currently indicated as a treatment for cyanide poisoning or for the production of a lotion containing sodium thiosulfate pentahydrate for the treatment of tinea versicolor. It has been recently reported that sodium thiosulfate pentahydrate is an effective treatment for calciphylaxis (Ackermann et al., Archives of Dermatology 2007, 143 (10): 1336-1337). It has also been reported that sodium thiosulfate pentahydrate is an effective treatment for vascular calcification (O'Neill, Kidney International 2008, 74 (11): 1376-1378). It has been reported that sodium thiosulfate pentahydrate is an effective treatment to prevent platinum-induced ototoxicity and nephrotoxicity that is associated with the use of platinum-containing chemotherapeutic agents (Skinner, Current Opinions in Oncology 1995, 7 (4): 310-315).

Sodium thiosulfate (STS) tetrahydrate also has been useful for treating platinum-induced ototoxicity, platinum-induced nephrotoxicity, cyanide poisoning, calciphylaxis, vascular calcification associated with atherosclerosis and fungal infection of the skin, nail beds or nails, as described in U.S. Pat. No. 8,496,973.

We have now found that topical, nasal or oral administration of sodium thiosulfate, with or without supportive cofactors, as will be discussed below, advantageously can be used for the treatment of various inflammatory, neurodegenerative and neuropsychiatric conditions by increasing reduced glutathione levels in the body. While not wishing to be bound by theory, it is believed administration of sodium thiosulfate can increase glutathione levels in the body to offset reduction in endogenous glutathione in patients suffering from various inflammatory, neurodegenerative and neuropsychiatric conditions.

Oxidative stress and inflammation are important pathogenic mechanisms involved in various inflammatory, neurodegenerative and neuropsychiatric conditions, such as, for example, Neuropsychiatric disorders, Pain disorders, Alzheimer's disease, Autism Spectrum Disorders, Parkinson's Disease, Amyotrophic lateral sclerosis, Mitochondrial Dysfunction, Multiple Sclerosis, Fibromyalgia, Psoriasis, Rheumatic Diseases, Mold toxicity and Acne.

One common finding in these conditions is the low available levels of endogenous reduced glutathione. Deficits in available reduced glutathione pools subject cellular structures to increased oxidative stress, inflammation, and environmental toxicity. Glutathione is a powerful antioxidant that plays a critical role in protecting cells from oxidative damage caused by free radicals and reactive oxygen species (ROS). When glutathione levels are low, cells are more vulnerable to oxidative stress, which can lead to inflammation. Several studies have shown that low glutathione levels are associated with increased inflammation.

Inflammation is the body's natural response to injury or infection, but chronic inflammation can contribute to a variety of health problems, including heart disease, diabetes, and cancer, as well as the conditions stated herein. When cells are exposed to oxidative stress, they can release pro-inflammatory molecules, e.g. cytokines, which can trigger inflammatory responses. Overall, maintaining adequate levels of glutathione is important for protecting cells from oxidative stress and reducing inflammation.

Mycotoxins can affect glutathione levels. Mycotoxins are toxic substances produced by certain mold species that can contaminate food, animal feed, and other agricultural products.

One of the ways that mycotoxins can cause oxidative stress is by depleting glutathione levels. When mycotoxins are present in the body, they can trigger the production of reactive oxygen species (ROS), which can cause oxidative damage to cells. In response to this oxidative stress, the body may use up its stores of glutathione in an attempt to neutralize the ROS and protect cells from damage. When mycotoxins are ingested, they can cause a variety of health problems, including liver damage, immune system suppression, and oxidative stress.

Evidence suggests that numerous mycotoxins are contaminating agricultural crops world wide, including but not limited to corn, peanuts, wheat, barley, soybeans, rice, rye, sorghum, oats as well as various fruit and vegetables. India, China and the United States have been shown to have the highest contamination of mycotoxins in crops worldwide, particularly corn.

Amongst the most prevalent contaminants in crops worldwide, besides mycotoxins, is the herbicide glyphosate, used to control weeds and other unwanted vegetation in agricultural and non-agricultural settings. Evidence suggests that glyphosate may reduce glutathione levels.

Glyphosate is one of the most widely used herbicides in the United States, and it is estimated that a significant percentage of crops are treated with this chemical. According to data from the United States Department of Agriculture (USDA), glyphosate use has steadily increased over the past few decades, and it is now the most commonly used herbicide in the country.

In 2016, the USDA reported that glyphosate was used on approximately 298 million acres of crops in the United States, which represents about 60% of the total cropland area. The crops most commonly treated with glyphosate include soybeans, corn, cotton, and wheat.

Glyphosate works by inhibiting the activity of an enzyme called EPSP synthase, which is involved in the production of certain amino acids that are necessary for the synthesis of proteins. This ultimately causes the plant to die. While glyphosate is considered safe when used according to label instructions, there is ongoing debate about its potential health effects. Some studies have suggested that glyphosate may have negative effects on human health, including a reduction in glutathione levels.

Sulfation has been found to be affected in various inflammatory and neuroinflammatory conditions, including Alzheimer's disease, Autism Spectrum Disorders, Parkinson's Disease, Amyotrophic lateral sclerosis, Mitochondrial Dysfunction, Multiple Sclerosis, Fibromyalgia, Psoriasis, Rheumatic Diseases and Acne. Sulfation is a process in which sulfate molecules are added to various compounds in the body, including hormones, neurotransmitters, and other signaling molecules. This process is important for regulating many physiological processes in the body, including detoxification, immune function, and brain development.

For example, several studies have found that individuals with autism have abnormalities in sulfation pathways. Research has shown that children affected with autism have lower levels of sulfate in their urine, which suggests that their sulfation pathways may be impaired. In addition, studies have found that individuals affected with autism have altered levels of several sulfated molecules, including hormones like melatonin and neurotransmitters like dopamine and serotonin. Impaired sulfation pathways may contribute to the development of autism by disrupting normal brain development and function. For example, sulfated molecules like melatonin and dopamine play important roles in regulating sleep, mood, and behavior, and alterations in these molecules may contribute to the behavioral symptoms associated with autism.

Several mycotoxins have been shown to affect sulfation pathways. For example, the mycotoxin ochratoxin A has been found to inhibit sulfation in the liver, which can lead to the accumulation of toxic compounds in the body. Similarly, another mycotoxin called deoxynivalenol (DON) has been shown to disrupt sulfation pathways in the intestine, which can impair immune function and increase susceptibility to infection. Studies have shown elevated levels of Ochratoxin A in patients affected with autism spectrum disorders. DON can enter the body through various pathways, including through our food supply. DON is one of the main concerns in all crops in North America and was present in 72% of corn samples and in 89% of cereal samples.

There is also evidence to suggest that glyphosate may affect sulfation in the body. Studies have shown that glyphosate exposure can disrupt sulfate metabolism, leading to alterations in the sulfation of proteins and other molecules. Glyphosate has also been shown to disrupt the gut microbiome, which can further impact sulfate metabolism and sulfation.

On the contrary, impaired sulfation pathways can also lead to increased susceptibility to mycotoxin toxicity. Sulfation is an important process for detoxifying many toxic compounds, including mycotoxins. When sulfation pathways are impaired, the body may be less able to remove mycotoxins from the body, leading to increased toxicity and health problems.

Depletion of glutathione (GSH) in glial cells induces neuroinflammation resulting in neuronal death. Neuroinflammation is, at least in part, characterized by the microglial release of proinflammatory factors such as cytokines and free radicals. Its purpose is to remove the source of harm so healing can take place. But when the inflammation is prolonged, it may cause neuronal dysfunction and death. Chronic neuroinflammation is closely associated with the pathogenesis of several neurodegenerative diseases, including but not limited to Alzheimer's disease (AD), Parkinson's disease (PD) as well as Autism.

Chronic oxidative stress results in elevations of oxidized to reduced glutathione ratio which inhibits methylation at various points, in particular the formation S-Adenosylmethionine (SAMe) through the enzyme S-Adenosylmethionine synthetase. In human physiology, this is a critical component of the Krebs cycle and metabolic dysfunction, as described, for example, in Autism Spectrum Disorders.

It is important to note that the accumulation of oxidized Glutathione (GSSG) and the inability to regenerate GSH can disrupt the cellular antioxidant defense system and impair the ability to counteract oxidative stress. This imbalance in the redox state can have detrimental effects on cellular functions and may contribute to various diseases and disorders associated with oxidative stress. If GSSG cannot be efficiently reduced back to GSH, it may accumulate within the cell. Accumulated GSSG can lead to an imbalance in the cellular redox state, resulting in increased oxidative stress and potential damage to cellular components, including proteins, lipids, and DNA. This oxidative stress can contribute to cellular dysfunction and may be associated with the pathological conditions mentioned herein.

STS can interact with glutathione disulfide (GSSG), which is the oxidized form of glutathione. The interaction occurs through a redox reaction where STS acts as a reducing agent.

When STS comes into contact with GSSG, it can donate sulfur molecules to reduce GSSG back to its reduced form, glutathione (GSH). This reaction converts the disulfide bond in GSSG into two molecules of GSH. The overall reaction can be represented as follows:

2 GSSG+Na2S2O3->2 GSH+Na2SO4

In this reaction, STS (Na2S2O3) donates sulfur (S2O3) to break the disulfide bond in GSSG, resulting in the formation of two molecules of reduced glutathione (GSH) and sodium sulfate (Na2SO4) as a byproduct.

This reduction reaction is important for maintaining the redox balance in cells and replenishing the pool of reduced glutathione. Glutathione plays a crucial role in antioxidant defense and cellular detoxification processes, and the regeneration of GSH from GSSG is essential for its proper functioning.

It's worth noting that this interaction between STS and GSSG is one of several ways in which GSSG can be reduced back to GSH. Cells have enzymatic systems, such as glutathione reductase, which facilitate the regeneration of GSH from GSSG. STS can serve as an exogenous reducing agent in certain experimental or clinical settings to promote GSSG reduction and maintain cellular redox homeostasis.

From additional studies it is also believed that administration of sodium thiosulfate can increase glutathione levels in the body. STS increases H2S (hydrogen sulfate) and GSH (glutathione) expression in human microglia and astrocytes. When human microglia and astrocytes are activated by lipopolysaccharide (LPS)/interferon-γ (IFNγ) or IFNγ, they release molecules that are toxic to differentiated human derived cell lines. When glial cells are treated with STS, there is a significant enhancement of neuroprotection. This at least is partially due to the antioxidative function of this agent; STS reacts with GSSG (oxidized glutathione) to produce reduced glutathione in the presence of hydroxyl radicals or peroxides. In addition, STS has a potential to produce hydrogen sulfide (H2S) by reaction with trans-sulphuration enzymes.

STS has been shown to reduce the toxicity of mycotoxins. In a 2018 study published in the Journal of Environmental Science and Health, researchers found that treatment with STS reduced the toxicity of the mycotoxin, deoxynivalenol (DON). STS was able to reduce the levels of oxidative stress and inflammation caused by DON exposure, which are some of the mechanisms that contribute to its toxicity.

STS is able to reduce the levels of several mycotoxins in corn and wheat samples, e.g. STS was able to significantly reduce the levels of aflatoxins, ochratoxin A, and zearalenone, which are some of the most common mycotoxins found in food products.

The present invention provides a formulation of key compounds, which include STS as the main active constituent that provides antioxidant support, detoxification of free radicals, recycling of oxidized glutathione to reduced glutathione, as well as cofactors for key enzymatic processes, considered critical, such as methylation pathways and immune mediators, which can assist the human metabolic function in said neurodegenerative dysfunction/inflammatory diseases and during periods of metabolic burden.

STS may be administered in topical, nasal, or oral form with or without various supportive cofactors, as will be described below.

According to one aspect of our invention we provide methods and compositions for treating mycotoxins from dietary contribution or produced from fungal infection, mold toxicity or acne.

In another aspect we provide methods and compositions for treating oxidative stress and neuroinflammation consistent with neurodegenerative disorders such as pain disorders, Alzheimer's disease, ALS, Autism Spectrum Disorder, Parkinson's Disease, allotropic lateral sclerosis (ALS), Mitochondrial Dysfunction, Multiple Sclerosis (MS) and Fibromyalgia, as well as neuropsychiatric conditions, and inflammatory conditions such as rheumatic diseases, acne and psoriasis.

In another aspect we provide methods and compositions for treating LPS induced inflammation, which activate microglial cells, deplete available reduced glutathione and perpetuate inflammatory cytokines.

In another aspect we provide methods and compositions for increasing available reduced glutathione and increasing the body's overall antioxidant response to oxidative stress.

In still another aspect we provide methods and compositions for improving cognition, awareness, and speech as measured by SRS2 social responsiveness scale SRS-2 as applied to autism spectrum disorder.

We also provide methods and compositions for supporting transmethylation pathway and Krebs cycle as described herein.

Additionally, we provide methods and compositions for treating mitochondrial dysfunction resulting from oxidative stress and metabolic dysfunction or impairment in the Krebs cycle, Transmethylation pathway, and sulfation pathway.

Further provided are methods and compositions for increasing levels of thiocyanate for the benefit of immune mediation and inflammation through use of sodium thiosulfate.

Further provided are methods and compositions for increasing oxygen carrying capacity of red blood cells by use of STS as a reducing agent assisting the conversion of methemoglobin to functional hemoglobin through reduction of oxidized iron.

As used herein, “compositions” shall include various forms of materials including, without limitation, pharmaceutical grade sodium thiosulfate, including liquid forms, dry powder forms, capsule, caplet and pill forms, syrups, pastes, gels, salves, lotion, patch compress and bandage forms.

Also as used herein “sodium thiosulfate” shall include various hydrated forms of sodium thiosulfate including STS pentahydrate, which is available commercially in grades ACS, Reagent and USP.

STS Pentahydrate provides a potent antioxidant effect in human physiology including metabolic improvements through increasing available reduced oxidized glutathione, limiting inhibition of the trans-sulphuration pathway, and S-Adenosylmethionine synthetase by oxidized glutathione, increased excretion of toxic metabolites such as mycotoxins or glyphosate of food origin or mycotoxins through internal fungal infection.

STS participates in the recycling of oxidized glutathione through conversion to sulfite by the enzyme sulfite oxidase (SO), and sulfite can further react with H2S to produce thiosulfate and water. This reaction is mediated by the enzyme sulfite reductase (SiR), which uses H2S as a substrate.

The H2S produced by the reaction between sulfite and H2S can act as a reducing agent and directly reduce oxidized glutathione (GSSG) back to its reduced form (GSH). Additionally, the formed H2S has been shown to have antioxidant properties and can scavenge reactive oxygen species (ROS), which can indirectly protect and further support glutathione function.

Sodium thiosulfate itself can scavenge free radicals and reactive oxygen species (ROS), which can cause oxidative damage to cells and disrupt the balance of antioxidants, including glutathione. By reducing the levels of ROS, STS indirectly helps preserve the pool of glutathione within cells.

The combined mechanisms of thiosulfate allow for multiple supportive pathways for the bolstering of bioavailable glutathione, which can contribute to redox balance within cells.

Glutathione is a tripeptide composed of three amino acids: cysteine, glycine, and glutamate. It plays a crucial role in maintaining cellular health by acting as a powerful antioxidant, detoxifying harmful compounds, regulating various cellular processes, playing a central role for detoxification, supporting methylation and sequestering of free radicals for conjugation and excretion from the body.

Additionally, STS can neutralize certain toxins or heavy metals that can deplete glutathione levels or impair its function. For example, STS is used as an antidote for cyanide poisoning because it reacts with cyanide to form thiocyanate, a less toxic compound. By counteracting the toxic effects of cyanide, STS helps maintain the integrity of cellular processes, including the proper functioning of glutathione.

It's important to note that while STS can indirectly support glutathione, the direct regulation and synthesis of glutathione are controlled by enzymatic processes within cells. Therefore, it is primarily through the reduction of oxidative stress and detoxification that STS can indirectly assist glutathione in carrying out its cellular functions.

STS additionally provides inhibition of the formation of methemoglobin, which can interfere with oxygen transport in the body. Sodium thiosulfate acts as a reducing agent, which helps convert methemoglobin back into functional hemoglobin by reducing the oxidized iron. By doing so, it can restore the oxygen-carrying capacity of the blood and improve oxygenation in affected individuals.

### BRIEF DESCRIPTION OF THE DRAWINGS

FIGS. 1-11 tabulate patient responses following administration of STS in accordance with the present disclosure.

### WORKING EXAMPLES

In accordance with the present invention, as embodied and broadly described in preferred embodiments herein, we use STS as the main active constituent in combination with cofactors as mentioned that assist in its role to provide support for, recycling of glutathione, methylation related to the Krebs cycle, trans-sulphuration pathway, and S-Adenosylmethionine synthetase, improvement in oxygen carrying capacity of red blood cells, and immune mediation via formation of thiocyanate.

Sodium thiosulfate alone or Sodium thiosulfate taken with identified cofactors in novel formulations are described below.

### Example 1

### Oral Administration

Sodium Thiosulfate may be administered with or without food but not on an empty stomach in a powder form dissolved in water or juice or capsule form swallowed with the following dosing:

Sodium Thiosulfate Pentahydrate [5-10 mg/kg×one to three times daily] With or without one or more of the following adjuvant cofactors given one to three times daily

Acetyl L Carnitine [5 mg/kg]
Beet Root Powder [3 mg/kg]
Alpha Keto Glutaric Acid [3 mg/kg]
NAC [3 mg/kg]
L glutamine [3 mg/kg]
Nicotinamide riboside [2-3 mg/kg]
Vitamin B3 as Niacinamide [2 mg/kg]
CoQ10 [2 mg/kg]
Zinc [0.5 mg/kg]
Vitamin B2 as riboflavin-5-phosphate [0.3 mg/kg]
Vitamin B6 as both Pyridoxal 5 phosphate and Pyridoxine HCL [0.3
mg/kg]
Methylcobalamin 5 mcg/kg
Cyanocobalamin 5 mcg/kg
Adenosylcobalamin 5 mcg/kg
Molybdenum Glycinate [2 mcg/kg]

Or in the following approximate mass ratio in the order of components listed above. 50:50:30:30:30:30:20:20:20:5:3:3:0.05:0.05:0.05:0.02

### Example 2

### Nasal Administration

Sodium Thiosulfate administered with or without food but not on an empty stomach in a solution of sodium chloride with the following concentrations of constituents:
10 mg/ml concentration of sodium thiosulfate pentahydrate
5 mg/ml concentration of sodium chloride

One spray of volume 1/10th of 1 ml in each nostril one to three times daily

### Example 3

### Topical Administration

Sodium Thiosulfate may be administered in a lotion as defined below 400 mg Sodium Thiosulfate per application dissolved in a lotion with the following base constituents to be applied with the following frequency per body weight topically recommended to the following bodily areas as depicted below.
Children 2-3 years old once daily
Children>15 kg 1-2 times daily
Children>20 kg 2-3 times daily
Children>30 kg 3 times daily

Lotion is defined as a constituent of the following products in order to provide an aqueous substrate for dissolved sodium thiosulfate
Constituent of products in a Typical Compatible lotion not limited to
Water, mineral oil, glycerin, cetyl alcohol, sorbitol, caprylic/capric triglyceride, emulsifying wax NF, stearic acid, dimethicone, phenoxyethanol, cetearyl alcohol, lecithin, carbomer, sodium hydroxide, ceteareth-20, diazolidinyl urea, sodium citrate, methylparaben, BHT, retinyl palmitate, sodium pyruvate, propylparaben, fragrance, citric acid, ethylparaben, panthenol, tocopheryl acetate,

With or without the following cofactors with the following dosing per 400 mg topical administration

| Units | Quantity | Component |
|---|---|---|
| IU | 500.00 | Vitamin E |
| IU | 200.00 | Vitamin A |
| mg | 0.33 | methylcobalamin |
| mg | 0.33 | Adenosylcobalamin |
| mg | 0.33 | Cyanocobalamin |
| IU | 600.00 | D3 |
| mg | 0.20 | K1 |
| mg | 0.67 | folinic acid |

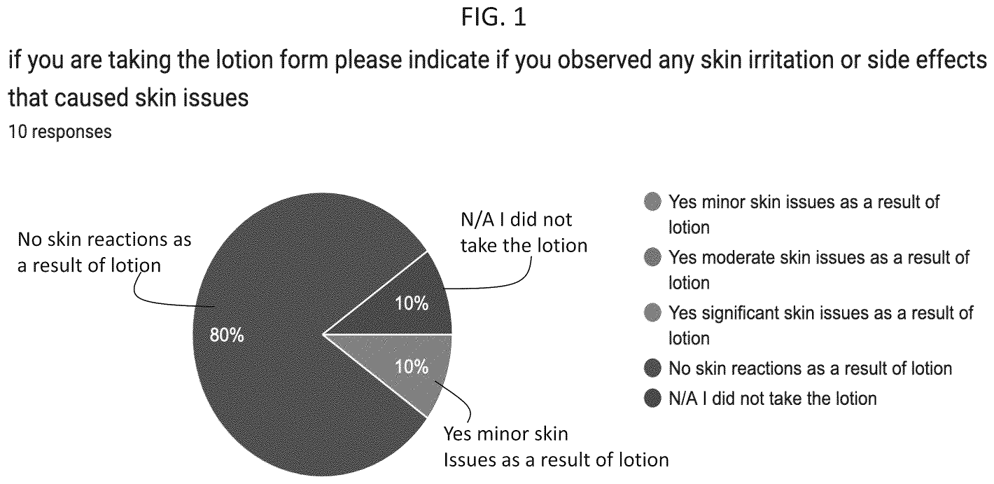

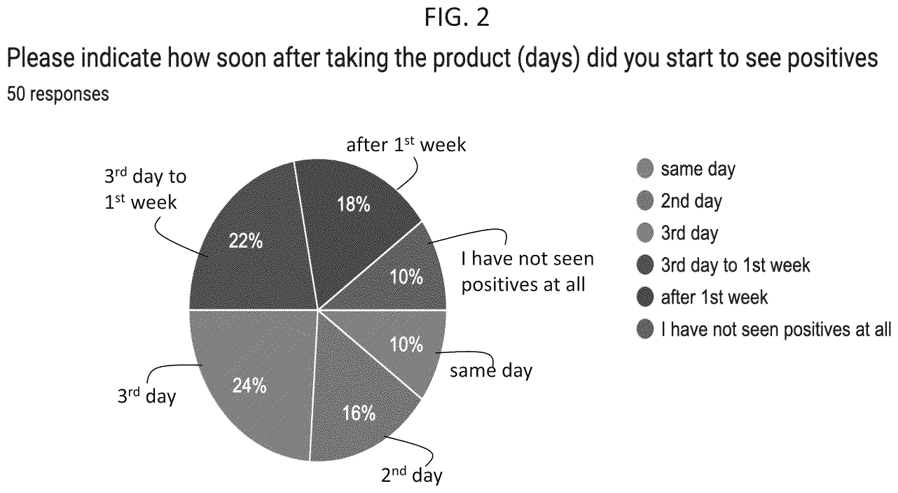

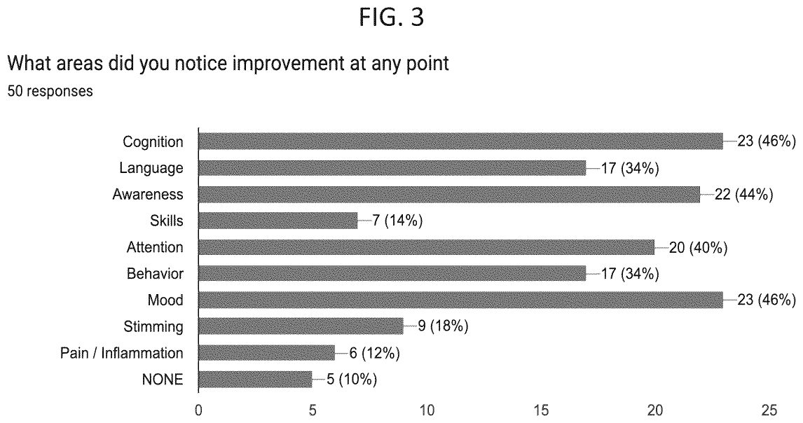

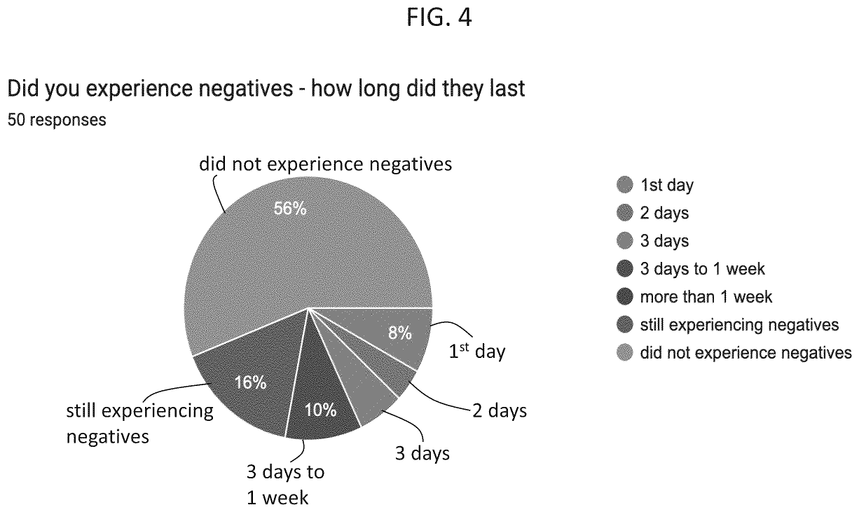

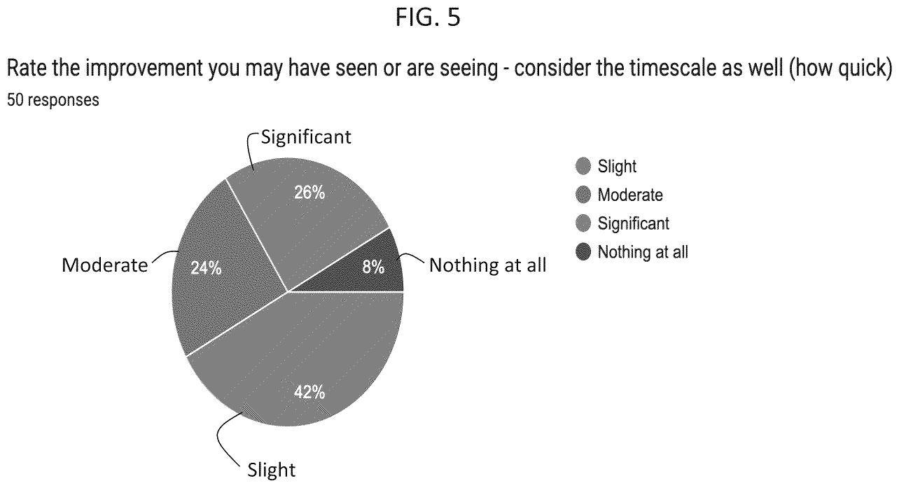

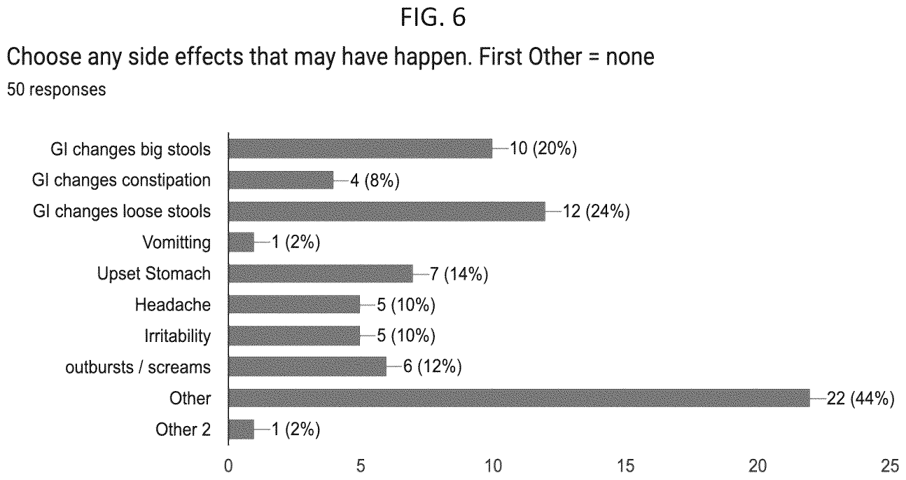

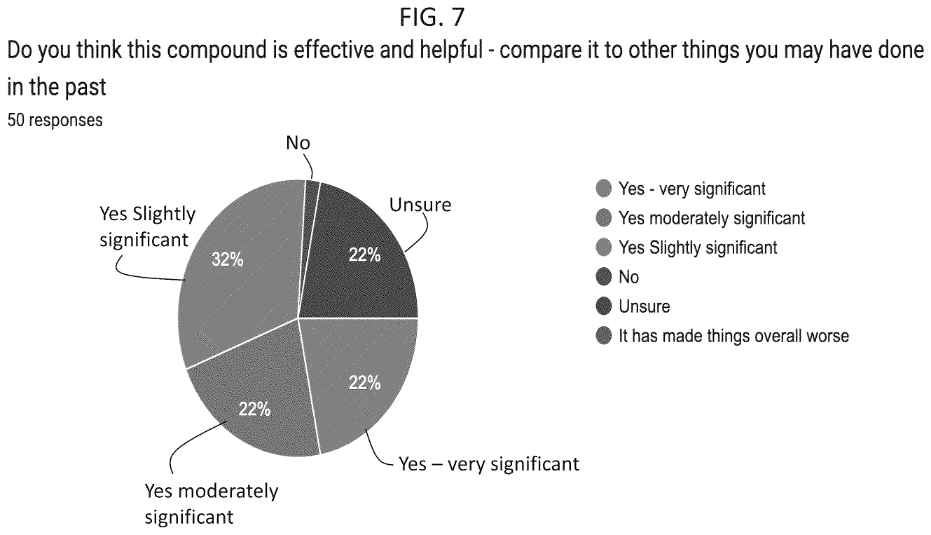

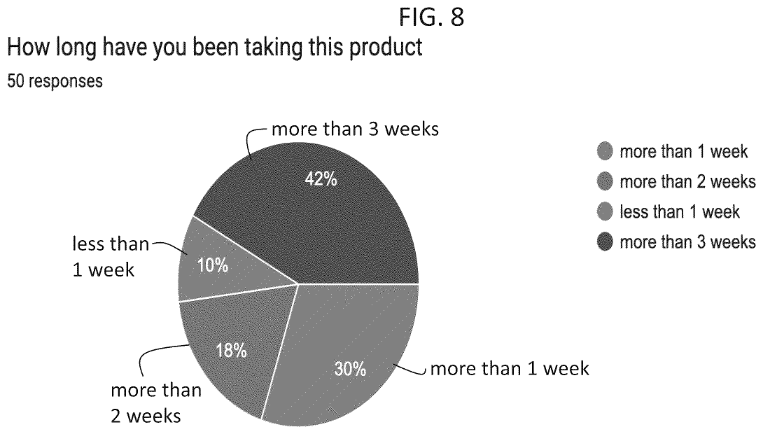

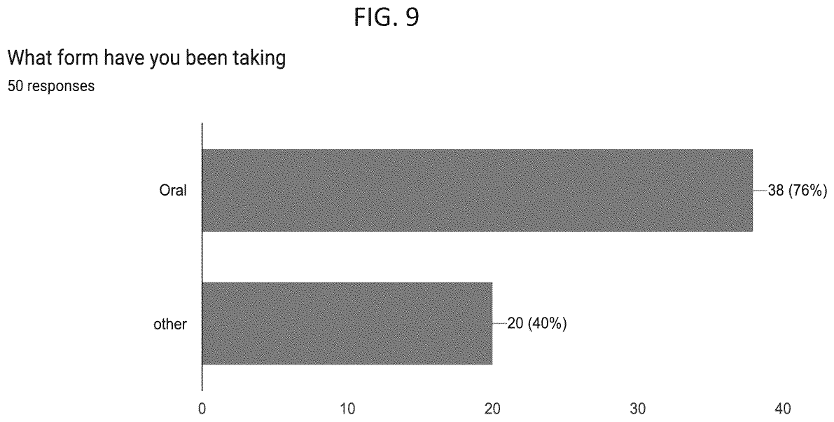

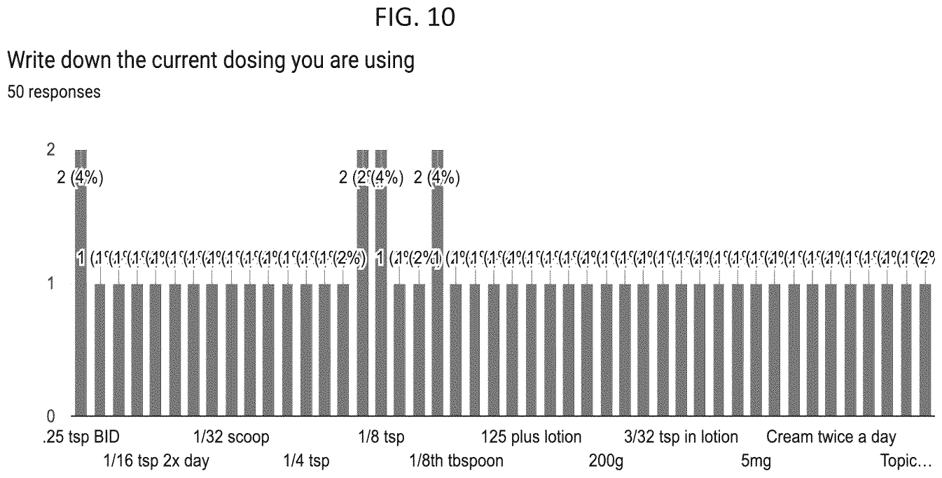

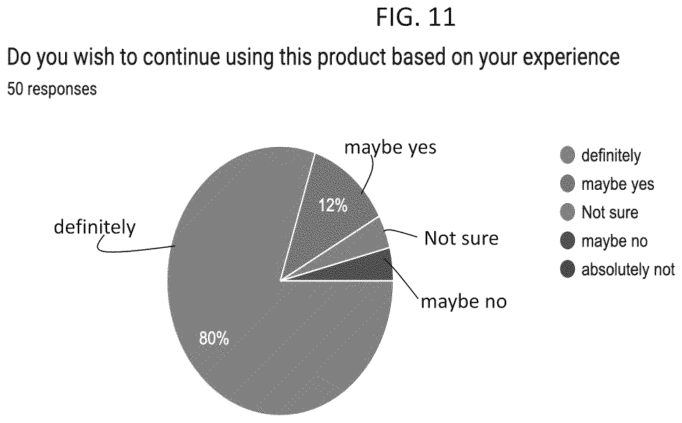

The following and FIGS. 1-11 tabulate patient responses/results:

Questionnaire Responses from 50 Patients (all Including the Conditions Mentioned Herein) Either Taking the STS in Cream or Oral Form:

Write down briefly changes you have observed and comments PLEASE DO NOT NAME THE COMPOUND in this questionnaire:
“Better sleep and better receptive instructions. Negative, mainly mood swing and uncontrolled peeing.”
“Increased awareness, reception, skills at therapy, increased sounds. We feel like he can rationalize better!”
“We observed reduction in his finger flicking and finger writing stimming for around 30 to 40%.”
“More calm, slightly more communication and longer sentences, slightly better eye contact.”
“For my son better communication, focus, attention, patience, self regulation for my daughter cognition, motor planning, expressive language, self regulation.”
“In combination with sporanox, I noticed more aware and more present.”
“Improved focus, more present,—seems to be more stimmy but hard to say if this is from footbaths—mobilizing toxins? or is this from the thio.”
“Better communication and interaction with others.”
“School says lots of language. I see appropriate play. Better eye contact. Response quicker when asked a question. Decreased stimming until day 10, then regressed.”
“Calmer, cognitive gains, more focused, better sleep, dreaming again after many years, belly more flat, more regular bathroom visits, less to no fatigue.”
“Slightly more fluid speech, improved attention and focus, better mood overall.”
“Better sleep, sleeping through the night.”
“Ability to say new words and approximate new sounds significantly, growing cognition, reduced stimming, increased awareness—in a very short period of time.”
“Attention, Language, cognition improved a lot, he is more aware and taking his and others responsibilities.”
“Improved attention and affection.”
“Slight improvement in behavior and mood.”
Better focus, more present, more energy, some cognitive function better, the will to want to do things, less bloating and gas.”
“Less stimming; more bowel movement.”
“No arthritic pain.”
“He had a really good day at school a day ago and was very communicative and happy at home.”
“Significant increase in energy and stamina-no more deep fatigue that I felt before. Also much calmer mentally and more focused.”
“First day of taking it, didn't feel well at all, ended up with strep throat he seems more aware than before, started clearly pronounce words.”
“A slight to moderate change in all areas. Calmer and showing affection. Responding to some verbal cues. More engaged and aware. Saying some new words. Appears inflammation has come down significantly.”
“Meltdowns are decreased in frequency and duration. Self injury is less severe and less frequent.”
“Mood is improved. Excess night time urination has stopped.”
“Less stimming, better eye contact and bowl movement.”
“Less noisy and happier.”
“Increased executive functioning and rash is going away.”
“Reduction of appetite.”
“He is responding to why question more relatively. E.g I don't want to do this because I am not a baby.”
“My son has been on the lotion for about a week. The first few days dysgraphia not mush changed. On the third day I saw an outburst of what seemed to be out of nowhere. My son was very sensitive to noise or anyone touching him. After that it has been a lot better. He seemed very aware of his surroundings. At times he's a lot calmer and less anxious about leaving the house and sleeping better.”
“Joint pain diminished specifically elbow, better cognition less fogginess if any.”
“Better mood, slightly more fluid speech.”
“No significant changes.”
“Behavior Less aggressive, slightly less obsessive and improved mood.”
“Stimming 80-90% reduced on day 1, calmer, more aware, happy, cuddly, smiling, even took a nap, which he never does because usually he is so hyper.”
“It definitely made me feel more alert and focused. However the more frequently I take it the less effective it seems to be. Or rather, I don't notice the alertness and focused-ness as much as I did when I first started taking it. Seems to have worn off a bit.”
“Increased focus and attention. Increased socialization and seeking interaction. Good mood.”
“Running nose has finally stopped after 3 months. Much happier; improved language with responding to yes/no questions-initiated going to the bathroom independently which he has never done before.”
“Was able to answer ‘why’ questions. Is generally more aware-help the door open for me after he went through it first for example.”
“More Language, wants to spend more time with family, overall happy and no aggression. After one day on the lotion woke up more awake and alert than she has been although had a little bit of a seizure-like behavior she hasn't had in a while. The oral form seemed to be initiate some biofilm release as well.”
Has been constipated for 2 weeks (stools in shape of small balls), keeps having stimming, teeth grinding.”
“No change.”
“More speech, interaction, cognition, concentration, better behaviour. Increase in mood and energy. Feel adhd symptoms improved related to motivation and not feeling overwhelmed by tasks.”
“More comprehension.”
“I had significant GI issues before starting the product. I noted some improvement after 2-3 days. It has steadily worsened and I am eliminating different supplements to see if anything is a factor. I believe food is an issue. I did Viome testing and must avoid some foods and take pre and pro biotics that they recommend. I am doing that now and have stopped the product.”

1: A method for treating a patient or subject suffering from autism spectrum disorder, the method comprising administering to the patient or subject a therapeutically effective amount of sodium thiosulfate pentahydrate, wherein the sodium thiosulfate pentahydrate is administered orally or nasally with a cofactor selected from the group consisting of Acetyl L Carnitine, Beet Root Powder, Alpha Keto Glutaric Acid, NAC, L glutamine, Nicotinamide riboside, Vitamin B3, Zinc, Vitamin B2, Vitamin B6, Methylcobalamin, Cyanocobalamin, Adenosylcobalamin, Molybdenum Glycinate, and a mixture thereof, or wherein the sodium thiosulfate pentahydrate is administered topically with a cofactor selected from the group consisting of Vitamin A, methylcobalamin, Adenosylcobalamin, Cyanocobalamin, folinic acid, and a mixture thereof.

2: The method of claim 1, wherein the sodium thiosulfate pentahydrate is administered in a pharmaceutically acceptable carrier.

3: The method of claim 1, wherein the sodium thiosulfate pentahydrate is administered orally.

4: The method of claim 1, wherein the sodium thiosulfate pentahydrate is administered nasally.

5: The method of claim 1, wherein the sodium thiosulfate pentahydrate is administered topically.

6-10. (canceled)

11: A method for treating a patient or subject suffering from mitochondrial disfunction, the method comprising administering to the patient or subject a therapeutically effective amount of sodium thiosulfate pentahydrate, wherein the sodium thiosulfate pentahydrate is administered orally or nasally with a cofactor selected from the group consisting of Acetyl L Carnitine, Beet Root Powder, Alpha Keto Glutaric Acid, NAC, L glutamine, Nicotinamide riboside, Vitamin B3, Zinc, Vitamin B2, Vitamin B6, Methylcobalamin, Cyanocobalamin, Adenosylcobalamin, Molybdenum Glycinate, and a mixture thereof, or wherein the sodium thiosulfate pentahydrate is administered topically with a cofactor selected from the group consisting of Vitamin A, methylcobalamin, Adenosylcobalamin, Cyanocobalamin, folinic acid, and a mixture thereof.

12: The method of claim 11, wherein the sodium thiosulfate pentahydrate is administered in a pharmaceutically acceptable carrier.

13: The method of claim 11, wherein the sodium thiosulfate pentahydrate is administered orally.

14: The method of claim 11, wherein the sodium thiosulfate pentahydrate is administered nasally.

15: The method of claim 11, wherein the sodium thiosulfate pentahydrate is administered topically.

16-17. (canceled)

18: A method for treating a patient or subject suffering from mold toxicity, the method comprising administering to the patient or subject a therapeutically effective amount of sodium thiosulfate pentahydrate, wherein the sodium thiosulfate pentahydrate is administered orally or nasally with a cofactor selected from the group consisting of Acetyl L Carnitine, Beet Root Powder, Alpha Keto Glutaric Acid, NAC, L glutamine, Nicotinamide riboside, Vitamin B3, Zinc, Vitamin B2, Vitamin B6, Methylcobalamin, Cyanocobalamin, Adenosylcobalamin, Molybdenum Glycinate, and a mixture thereof, or wherein the sodium thiosulfate pentahydrate is administered topically with a cofactor selected from the group consisting of Vitamin A, methylcobalamin, Adenosylcobalamin, Cyanocobalamin, folinic acid, and a mixture thereof.

19: The method of claim 18, wherein the sodium thiosulfate pentahydrate is administered in a pharmaceutically acceptable carrier.

20: The method of claim 18, wherein the sodium thiosulfate pentahydrate is administered orally.

21: The method of claim 18, wherein the sodium thiosulfate pentahydrate is administered nasally.

22: The method of claim 18, wherein the sodium thiosulfate pentahydrate is administered topically.

23-24. (canceled)
